(19) United States
(12) Patent Application Publication
(10) Pub. No.: US 2005/0131407 A1
(43) Pub. Date: Jun. 16, 2005

(54) Flexible spinal fixation elements

(76) Inventors: Christopher W. Sicvol, Boston, MA (US); Michael Mahoney, Middletown, RI (US); Riley Hawkins, Cumberland, RI (US); James Brennen, Florence, SC (US); Carl Lauryssen, Malibu, CA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/737,734

(22) Filed: Dec. 16, 2003

(51) Int. Cl. A61B 17/56; A61B 17/58

(52) U.S. Cl. 606/61

(57) ABSTRACT

A flexible spinal fixation element is provided that is movable between a first position, in which the spinal fixation element is adapted to be angularly manipulated, and a second, locked position, in which the spinal fixation element is aligned in a desired orientation and is immovable. The configuration of the flexible spinal fixation element can vary, but the fixation element is preferably formed from a bioimplantable member having segments or a bellows configuration that allows the fixation element to be selectively configurable between the first and second positions. In use, the flexibility of the spinal fixation element allows the fixation element to be introduced through a percutaneous access device, thereby advantageously allowing the fixation element to be implanted using minimally invasive techniques.

### FIELD OF THE INVENTION

This application relates to tools for use in spinal surgery, and in particular to a spinal fixation element that is flexible prior to locking, and methods for implanting the same.

### BACKGROUND OF THE INVENTION

Spinal fusion is a procedure that involves joining two or more adjacent vertebrae with a bone fixation device so that they no longer are able to move relative to each other. For a number of known reasons, spinal fixation devices are used in orthopedic surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. The fixation elements can have a predetermined contour that has been designed according to the properties of the target implantation site, and once installed, the instrument holds the vertebrae in a desired spatial relationship, either until desired healing or spinal fusion has taken place, or for some longer period of time.

Recently, the trend in spinal surgery has been moving toward providing minimally invasive devices and methods for implanting spinal fixation devices. The use of rigid, generally elongate spinal fixation elements, however, can be difficult to implant using minimally invasive techniques. One such method, for example, is disclosed in U.S. Pat. No. 6,530,929 of Justis et al., which utilizes two percutaneous access tubes for introducing an anchoring device, such as a spinal screw, into adjacent vertebrae. A spinal rod is then introduced through a third incision a distance apart from the percutaneous access sites, and the rod is transversely moved into the rod-engaging portion of each spinal screw. The percutaneous access tubes can then be used to apply closure mechanisms to the rod-engaging heads to lock the rod therein. While this procedure offers advantages over prior art invasive techniques, the transverse introduction of the rod can cause significant damage to surrounding tissue and muscle. Moreover, the use of three separate access sites can undesirably lengthen the surgical procedure.

Accordingly, there remains a need for improved minimally invasive devices and methods for introducing a spinal fixation element into a patient's spine.

### SUMMARY OF THE INVENTION

The present invention generally provides a spinal fixation element that is formed from an elongate, bioimplantable member having at least two segments that are selectively movable with respect to one another. As a result, the elongate member is configurable in a first, flexible position, in which the segments are adapted to be angularly manipulated with respect to one another, and a second, locked position, in which the segments are aligned in a desired orientation and are immovable with respect to one another. Each segment preferably has a shape that is adapted to prevent movement between the segments when the segments are in the second, locked position.

The segments can have a variety of configurations, and in one embodiment, each segment can include a female end and an opposed male end such that the female end of each segment is adapted to nest the male end of an adjacent segment. In another embodiment, each segment has a substantially tubular shape with a concave end and an opposed convex end such that the concave end of each segment is adapted to nest the convex end of an adjacent segment. In yet another embodiment, every other segment preferably has a substantially spherical shape and intervening segments have a substantially tubular shape with opposed ends that are adapted to seat the spherical segments.

In other aspects of the invention, the elongate body can include at least two elongate segments that are mated to one another at an end thereof by a hinge. A sleeve member can be disposed around the hinge to maintain the elongate body in the second, locked position. Alternatively, or in addition, the device can include a locking mechanism that is adapted to mate to the hinge to maintain the elongate body in the second, locked position.

The present invention also provides a spinal fixation element that is formed from an elongate body that includes first and second separate segments. Each segment can be in the form of a generally elongate, hemi-spherical rod having two portions connected to one another at an end thereof by a hinge, and the hinge on each of the first and second separate segments is preferably configured to maintain the elongate body in the second, locked position when the first and second separate segments are placed together to form a cylinder.

In another embodiment, a spinal fixation element is provided having a flexible elongate cable, and a bioimplantable, generally elongate member slidably disposed around the cable. The elongate member is configurable in a first, flexible position, in which the member is adapted to be manipulated in multiple angular orientations, and a second, locked position, in which the member is fully compressed and it is immovably aligned in a desired orientation. In exemplary embodiment, the generally elongate member is a bellows, and more preferably opposed terminal ends of the bellows are adapted to seat a portion of a spinal anchor.

The present invention also provides a spinal implant kit that includes a percutaneous access tube having an inner lumen extending between proximal and distal ends, and a selectively flexible spinal fixation element that is configurable in a bendable position, in which the flexible spinal fixation element can be inserted through the lumen in the percutaneous access tube and angularly manipulated as it exits from the percutaneous access tube, and a locked position, in which the flexible spinal fixation element is compressed to be immovably aligned in a desired orientation.

Methods for implanting a flexible spinal fixation element are also provided.

### BRIEF DESCRIPTION OF THE DRAWINGS

FIG. 1 is a side perspective view of one embodiment of a flexible spinal fixation element, in the expanded position, coupled to two spinal screws;

FIG. 2 is a side perspective view of the spinal fixation element and spinal screws of FIG. 1 with the spinal fixation element in a locked position;

FIG. 3 is a top perspective view of the spinal fixation element and spinal screws shown in FIG. 2 in a curved configuration;

FIG. 4A is a side perspective view of a flexible spinal fixation element disposed over a cable in accordance with another embodiment of the present invention;

FIG. 4B is a side perspective view of the flexible spinal fixation element of FIG. 4A in the locked position;

FIG. 5 is a cross-sectional view of yet another embodiment of a flexible spinal fixation element in accordance with the present invention;

FIG. 6A is a side perspective view of another embodiment of a flexible spinal fixation element in accordance with the present invention;

FIG. 6B is a side perspective view of the flexible spinal fixation element of FIG. 6A and a sleeve adapted to be disposed over the fixation element to maintain the fixation element in a locked position;

FIG. 7A is a side perspective view of yet another embodiment of a flexible spinal fixation element according to the present invention;

FIG. 7B is a side perspective view of the flexible spinal fixation element of FIG. 7A in the locked position;

FIG. 8A is a side perspective view of a bellows-type flexible spinal fixation element in accordance with yet another embodiment of the present invention;

FIG. 8B is a side perspective view of the flexible spinal fixation element of FIG. 8A in a locked configuration;

FIG. 9A is a side perspective view of a first percutaneous access device mated to a first spinal screw, and a cut-away view of a second percutaneous access device mated to a second spinal screw and having a flexible spinal fixation element extending therethrough;

FIG. 9B illustrates the flexible spinal fixation element of FIG. 9A extending distally through the percutaneous access device;

FIG. 9C illustrates the flexible spinal fixation element of FIG. 9B extending between the adjacent spinal screws; and

FIG. 9D is a cross-sectional view of a portion of the spinal screws shown in FIG. 9C having the spinal fixation element extending therebetween and having a cable mated thereto.

### DETAILED DESCRIPTION OF THE INVENTION

The present invention generally provides a spinal fixation element that is movable between a first position, in which the spinal fixation element is adapted to be angularly manipulated, and a second, locked position, in which the spinal fixation element is aligned in a desired orientation and is immovable. The configuration of the spinal fixation element can vary, but the fixation element is preferably formed from a bioimplantable member having segments or a bellows configuration that allows the fixation element to be selectively configurable between the first and second positions. In use, the flexibility of the spinal fixation element allows the fixation element to be introduced through a percutaneous access device, thereby advantageously allowing the fixation element to be implanted using minimally invasive techniques.

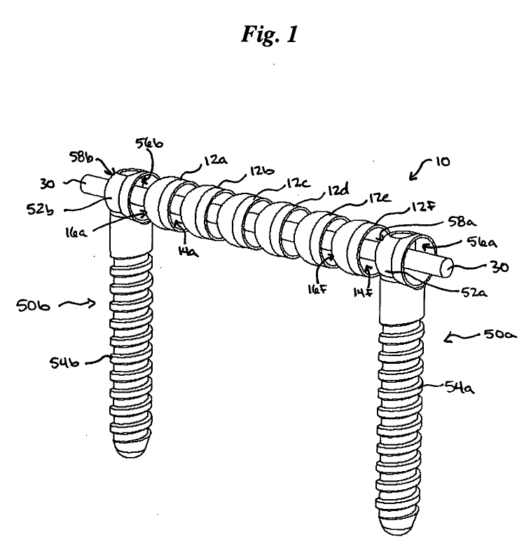

In one embodiment of the present invention, shown in FIGS. 1-5, the spinal fixation element can be formed from two or more segments that are slidably disposed around a cable. The cable, which serves as a guide wire for receiving and percutaneously delivering the segments to adjacent spinal anchors, allows the segments to be individually introduced into the surgical site, or to be angularly manipulated with respect to one another as they are implanted. Once the segments are positioned between adjacent spinal anchors, they can then be compressed or otherwise brought together to form a rigid spinal fixation element. The configuration, shape, and/or size of each segment is preferably selected to allow the segments to be locked into a desired configuration with respect to one another.

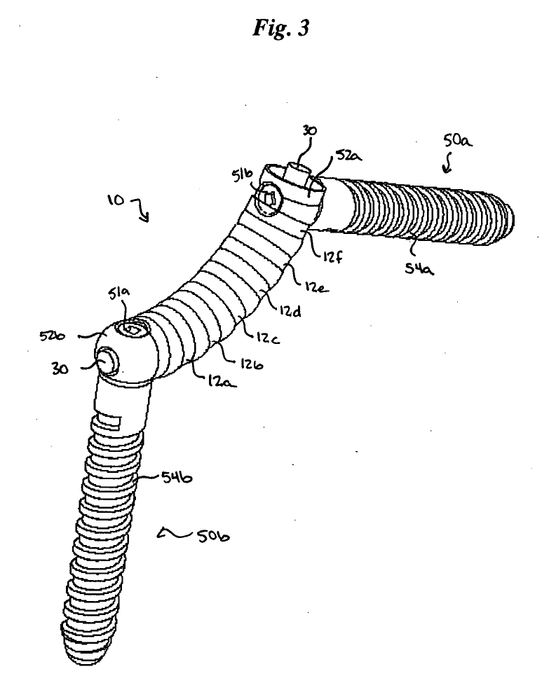

In the embodiment illustrated in FIGS. 1-3, the spinal fixation element 10 includes several segments 12a-12f, each of which is substantially cup-shaped and is slidably disposed around a cable 30. The cup-shape of the segments 12a-12f is such that each segment 12a-12f includes a first end 14a-14f having a substantially hollow, concave shape, and a second end 16a-16f having a substantially convex shape. This configuration allows the segments 12a-12f to be aligned along the cable 30 in the same direction so that the hollow, concave end 14a-14f of each segment receives or nests the convex end 16a-16f of the adjacent segment 12a-12f. The concave and convex configuration of the segments 12a-12f is particularly advantageous in that it allows the desired orientation of the fixation element 10 to be selectively adjusted, for example, to have a curved configuration, as shown in FIG. 3.

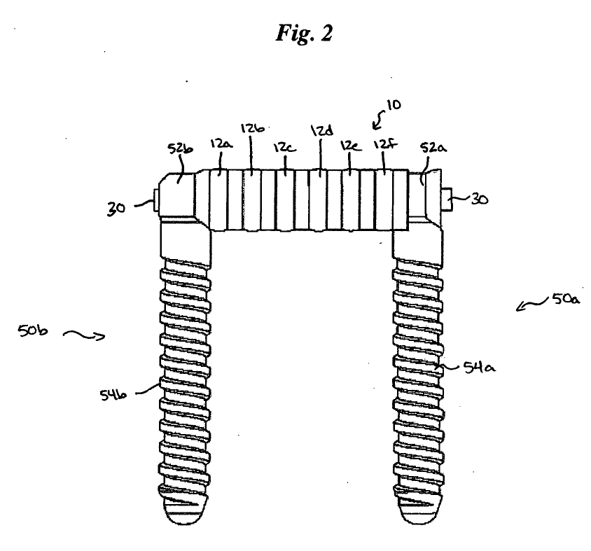

In use, the segments 12a-12f can be compressed between adjacent spinal anchors, such as spinal screws 50a and 50b, to lock the segments 12a-12f with respect to one another, thereby forming a rigid spinal fixation element 10, as shown in FIG. 2. In an exemplary embodiment, the terminal segments, i.e., segments 12a and 12f, are adapted to receive, or be received by, the head 52a, 52b of each screw 50a, 50b. In the embodiment shown in FIGS. 1-3, the screw heads 52a, 52b each have a shape that substantially corresponds to the shape of the segments 12a-12f so that the heads 52a, 52b form the terminal ends of the spinal fixation element 10 when the segments 12a-12b are compressed therebetween. Compression of the segments 12a-12f can be achieved by forcing the spinal screws 50a, 50b toward one another, as will be discussed in more detail below. Once the segments 12a-12f are formed into a spinal fixation element 10 and positioned in the desired configuration, the ends of the cable 30, which extend through the head 52a, 52b formed on each adjacent spinal screw 50a, 50b, can be locked into the head 52a, 52b using a closure mechanism, such as, for example, a set screw 51a, 51b (FIG. 3), that is threaded into each head 52a, 52b.

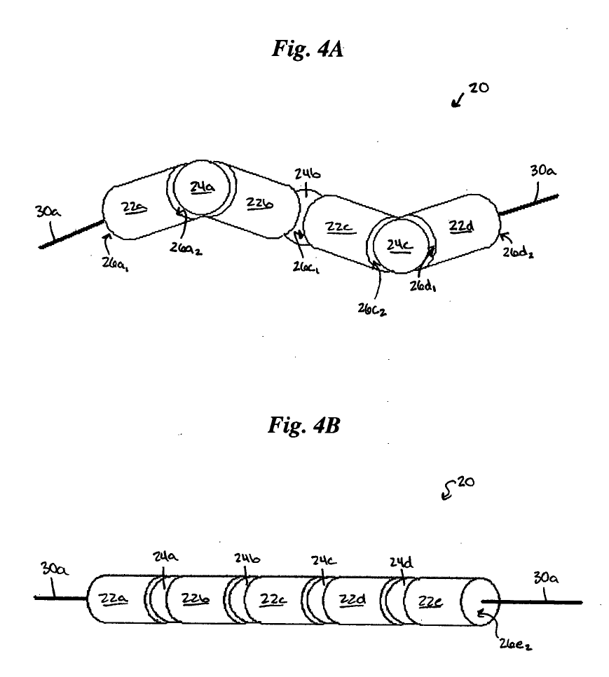

FIG. 4A illustrates another embodiment of a spinal fixation element 20 having segments 22a-22d, 24a-24c that are slidably disposed along a cable 30a, and in use, as shown in FIG. 4B, the segments 22a-22e, 24a-24d (FIG. 4B illustrates two additional segments) are adapted to lock together to form a rigid spinal fixation element 20. In this embodiment, segments 22a-22e have a substantially tubular shape with opposed first and second concave ends 26a₁-26e₁, 26a₂-26e₂, and the intervening segments 24a-24d are substantially spherical. As a result, the concave ends 26a₁-26e₁, 26a₂-26e₂ of the tubular segments 22a-22e will seat or nest the spherical segments 24a-24d to form a rigid spinal fixation element 20 when the segments 22a-22d, 24a-24e are compressed between adjacent spinal anchors. As previously stated with respect to FIGS. 1-3, the anchors and/or the terminal end segments, i.e., segments 22a and 22e in FIG. 4B, should have complementary configurations such that the receiver heads on the adjacent anchors form the terminal end segments of the fixation element 20. Thus, in the embodiment shown in FIGS. 4A-4B, for example, the receiver head of each anchor (not shown) should have a substantially spherical shape. Each head should also be adapted to receive the cable 30a and to receive a closure mechanism that is effective to lock the cable 30a in each head.

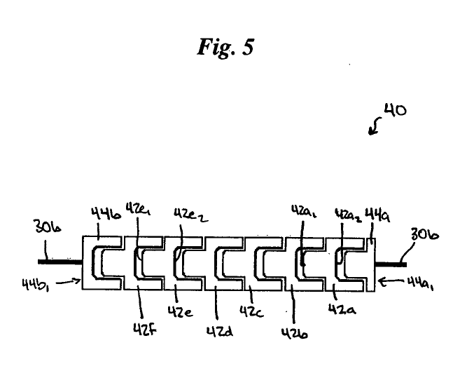

In yet another embodiment, shown in FIG. 5, the segments that form the spinal fixation element can include complementary male and female ends that are adapted to receive and/or mate to one another. As shown, each segment 42a-42e, which is slidably disposed around a cable 30b, includes a first, leading male end 42a₁-42e₁ and a second, trailing female end 42a₂-42e₂. The segments 42a-42e are aligned along the cable 30b in the same direction so that the trailing female end 42a₂-42e₂ of each segment 42a-42e receives the leading male end 42a₁-42e₁ of the next adjacent segment 42a-42e. The size of the male and female ends 42a₁-42e₁, 42a₂-42e₂ of the segments 42a-42e is preferably adapted to form a tight fit, e.g., a press-fit, therebetween, thus allowing the segments 42a-42e to be locked with respect to one another.

In order to lock the segments 42a-42e between the receiver heads of adjacent spinal anchors, the heads of the anchors can optionally include a male or female component for mating with the segments 42a-42e, or alternatively the terminal segments, e.g., segments 44a, 44b can be adapted to be positioned between the heads of the anchors. As shown in FIG. 5, the terminal segments 44a, 44b each include a substantially flattened terminal end surface 44a₁, 44b₁. While not shown, this surface 44a₁, 44b₁ can, however, have a shape that corresponds to an outer surface of the heads of the adjacent anchors. Again, the anchor receiver heads should be configured to receive a closure mechanism to secure the cable therein, thus locking the segments 42a-42e therebetween.

While the segments shown in FIGS. 1-5 can be locked together by a press-fit that is formed from compression of the segments between the heads of adjacent spinal anchors, the segments can optionally include features to facilitate the locking engagement therebetween. The concave ends 26a₁-26e₁, 26a₂-26e₂ of the tubular segments 22a-22e and/or the a portion or all of the spherical segments 24a-24d shown in FIGS. 4A-4B, for example, can include surface features formed thereon to prevent slippage between the segments 22a-22d, 24a-24e. The surface features (not shown) can be formed from a knurled surface, surface protrusions, a coating (e.g., a polymeric coating), or any other technique that will facilitate engagement between the segments 22a-22d, 24a-24e. In another embodiment, the segments can be configured to removably engage one another using, for example, a snap-fit. A person skilled in the art will appreciate that a variety of techniques can be used to provide a locking engagement between the segments.

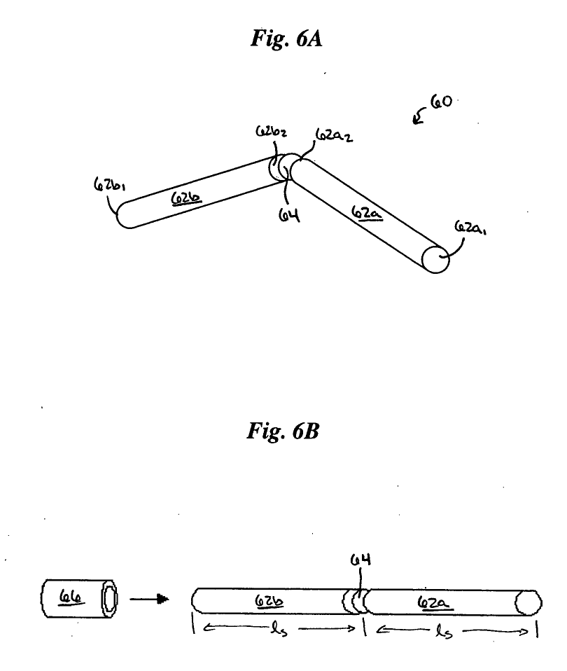

FIGS. 6A-8B illustrate additional embodiments of spinal fixation elements in accordance with the present invention. As with the fixation elements shown in FIGS. 1-5, each of the spinal fixation elements illustrated in FIGS. 6A-8B is configurable between a first, flexible position, and a second position in which the fixation element can be locked into a desired configuration.

Referring now to FIGS. 6A-6B, the spinal fixation element 60 includes first and second segments 62a, 62b that are mated to one another by a hinge 64. Each segment 62a, 62b can have any shape and size, but preferably each segment 62a, 62b has a generally cylindrical, elongate shape that allows the fixation element 60 to be used in place of traditional spinal rods. The hinge 64 is disposed between terminal ends 62a₂, 62b₂ of the segments 62a, 62b, and it allows the segments 62a, 62b to pivot with respect to one another. This is particularly advantageous in that the fixation element 60 can be introduced into adjacent spinal anchors through a percutaneous access tube, as the hinge 64 allows the segments 62a, 62b to bend with respect to one another. A person skilled in that art will appreciate that, in order to introduce the fixation element 60 through a percutaneous access device, each segment should have a length Is that is small enough to permit percutaneous access.

Once the fixation element 60 is positioned between adjacent spinal anchors, with terminal ends 62a₁, 62b₁ disposed within receiver heads of the adjacent anchors, a sleeve 66 or similar device can be disposed over the hinge 64 to prevent further bending of the segments 62a, 62b, thereby locking the segments 62a, 62b with respect to one another. Alternatively, or in addition, a screw of other locking mechanism can be applied to the hinge 64 to prevent further bending of the hinge 64. In another embodiment, where three spinal anchors are used, the hinge 64 can be positioned and locked within a receiver head of the middle spinal anchor, and the terminal ends 62a₁, 62b₁ can be disposed within adjacent spinal anchors. While only one hinge 64 is shown, a person skilled in the art will appreciate that the fixation element 60 can include any number of segments and hinges.

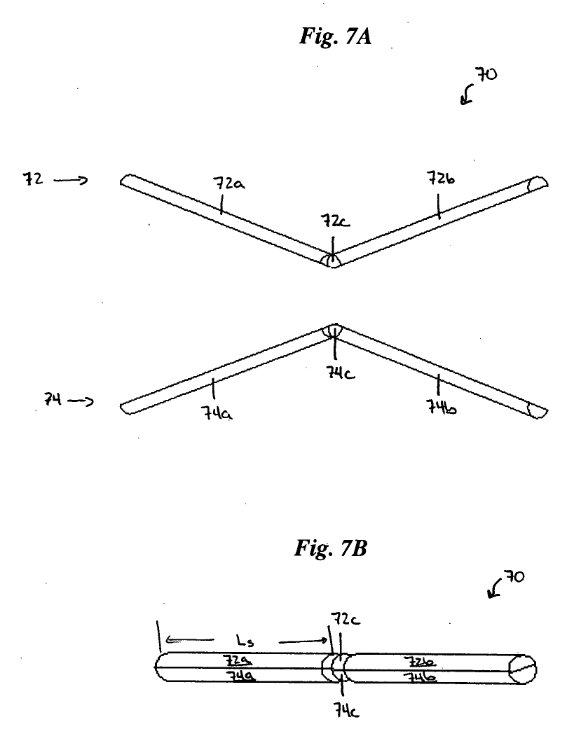

In yet another embodiment, shown in FIGS. 7A-7B, the spinal fixation element 70 can be formed from two separate segments 72, 74, each of which includes two portions 72a, 72b, 74a, 74b that are mated to one another by a hinge 72c, 74c. The segments 72, 74 are preferably configured such that the hinges 72c, 74c prevent one another from bending when the segments 72, 74 are joined and locked at opposed ends to form a spinal rod 70. In the illustrated embodiment, for example, segment 72 is formed from two portions 72a, 72b, each having an elongate, hemi-spherical shape. The hinge 72c is configured to allow the segments 72a, 72b to bend only uni-directionally. Segment 74 is similarly formed from two portions 74a, 74b, each having an elongate, hemi-spherical shape. The hinge 74c between portions 74a, 74b, however, is configured to allow the segments 72a, 72b to bend toward one another in a direction that is opposite to the direction that segments 72a, 72b bend. As noted above with respect to fixation element 60, the segments 72, 74 also preferably have a length Lₛ that allows the fixation element 70 to be percutaneously implanted.

In use, each segment 72, 74 can be introduced, preferably percutaneously, into a surgical site and positioned to extend between adjacent spinal anchors. The segments 72, 74 are positioned so that the hemi-spherical segments 72, 74, when placed together, form a single, cylindrical elongate rod 70. As a result, the hinges 72c, 74c prevent one another from bending, thus forming a rigid spinal rod 70. The terminal ends of the fixation element 70 can be locked into receiver heads of adjacent spinal anchors using techniques known in the art.

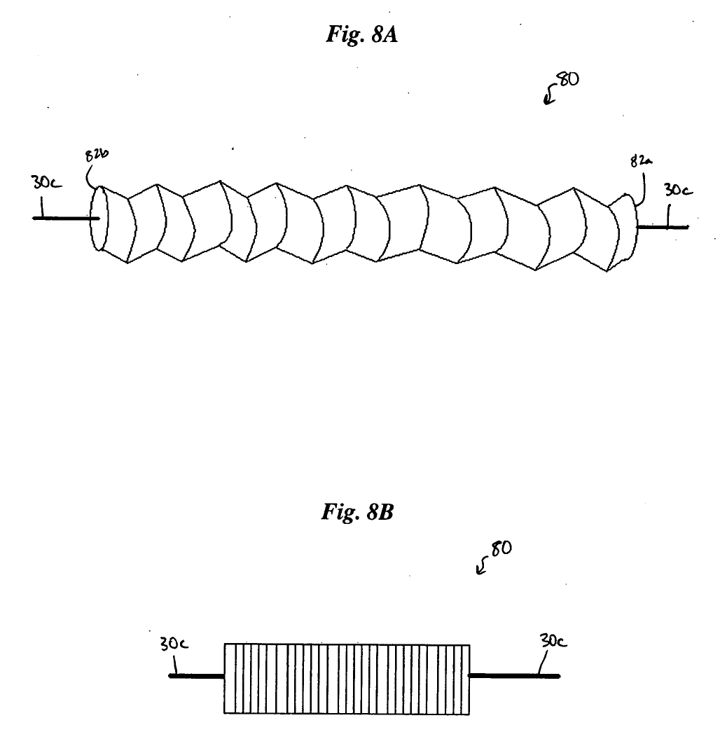

In another embodiment of the present invention, the spinal fixation element can be in the form of a bellows 80, as shown in FIGS. 8A and 8B. The bellows configuration of the fixation element 80 allows the fixation element 80 to be angularly manipulated as it is introduced into a surgical site and positioned between adjacent spinal anchors. The terminal ends 82a, 82b of the fixation element 80 are preferably adapted to seat the head of a spinal anchor, and thus they should have a shape that conforms to the shape of an outer surface of a spinal anchor head. Once positioned between adjacent anchors, the fixation element 80 can be locked at a desired orientation by compressing the bellows, as shown in FIG. 8B, and locking the cable 30c, which extends through the bellows 80, to the adjacent anchors.

A person skilled in the art will appreciate that the spinal fixation element of the present invention can have a variety of other configurations to allow the fixation element to be movable between a first position, in which the fixation element can be angularly manipulated, and a second position, in which the fixation element can be locked into a desired orientation.

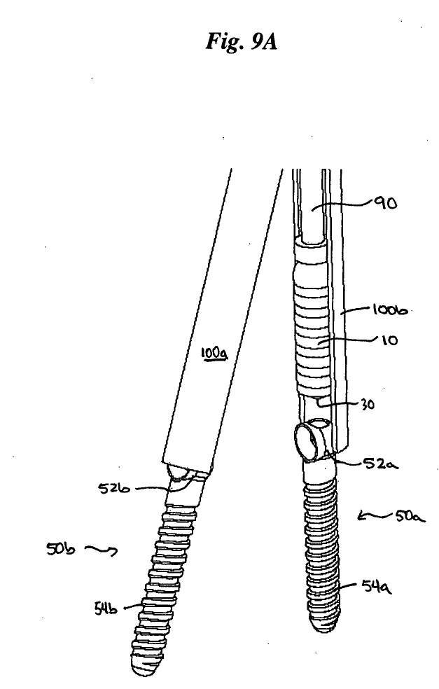

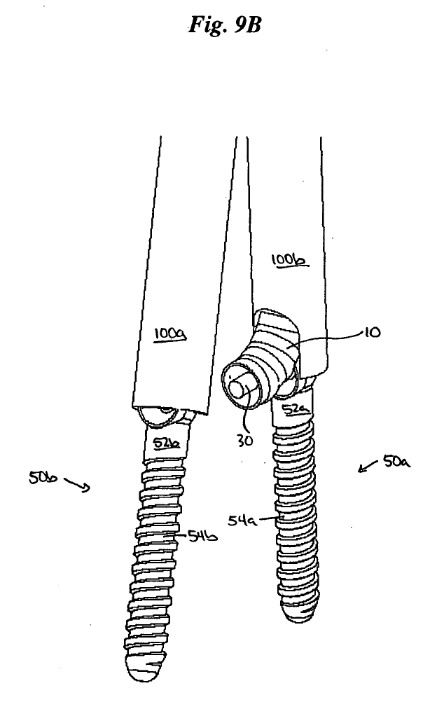

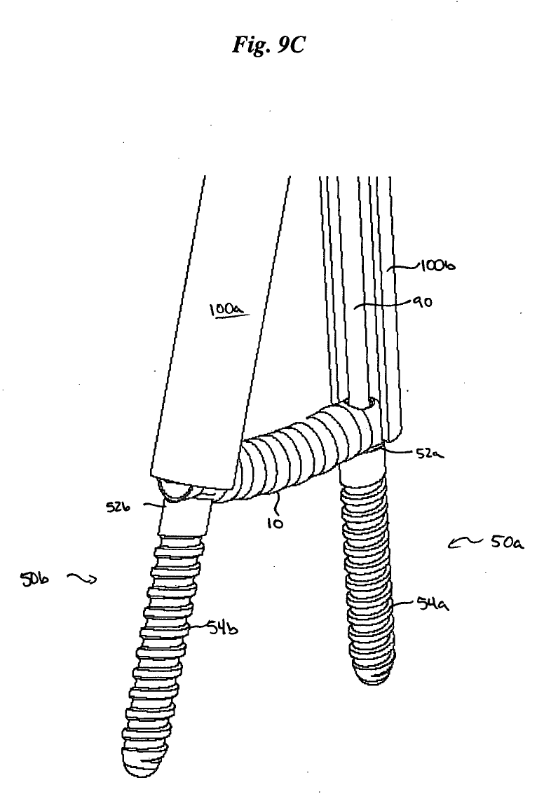

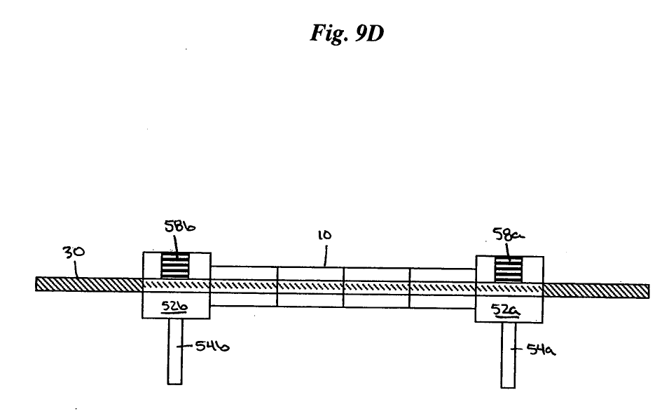

FIGS. 9A-9D illustrate an exemplary method of implanting a spinal fixation element using minimally invasive surgical techniques in accordance with the present invention. Fixation element 10 shown in FIGS. 1-3 is shown for illustration purposes only, and a person skilled in the art will appreciate that the method can be performed using any suitable spinal fixation element.

Referring to FIGS. 9A and 9B, two or more spinal anchors, e.g., spinal screws 50a, 50b, are implanted in adjacent vertebrae (not shown). While spinal screws 50a, 50b are shown, a variety of spinal anchors can be used with the present invention. As is further shown, each anchor has a percutaneous access tube 100a, 100b mated thereto. The spinal fixation element 10, tubes 100a, 100b, and/or anchors 50a, 50b can optionally be provided as part of a spinal kit. The anchors 50a, 50b, percutaneous access tubes 100a, 100b, and methods for implanting the same are described in more detail in a patent application filed concurrently herewith and entitled “Methods and Devices for Minimally Invasive Spinal Fixation Element Placement,” which is incorporated by reference herein in its entirety.

Once the spinal screws 50a, 50b are implanted with the tubes 100a, 100b attached thereto, the spinal fixation element 10 is introduced into one of the tubes, e.g., tube 100b, and it is advanced distally toward spinal screw 50a. A pusher shaft 90 can optionally be used to advance the fixation element 10 toward the anchor 50. In this embodiment, the spinal fixation element 10 is disposed around a cable 30. Thus, while not shown, the cable 30 is preferably advanced through the percutaneous access tube 100b and positioned to extend between the heads 52a, 52b of the adjacent anchors 50a, 50b prior to advancing the spinal fixation element 10 toward the anchor 50. The leading end of the cable 30 can optionally be locked into head 52b of anchor 50b, and the remaining portion of the cable 30 can serve as a guide cable. The fixation element 10 can then be passed along the cable 30, either as a whole or as individual segments, until the fixation element 10 is positioned between the heads 52a, 52b of the adjacent anchors 50a, 50b, as shown in FIG. 9C.

Once properly positioned, the percutaneous access tubes 100a, 100b can optionally be compressed toward one another using, for example, medical pliers, to compress the fixation element 10 between the adjacent anchors 50a, 50b. A closure device, such as a set screw, can then be introduced into the head 52a, 52b of each anchor 50a, 50b, or into the head of anchor 50a if anchor 50b already includes a closure mechanism, to lock the cable 30 thereto, as shown in FIG. 9D. The locking of the cable 30 between the adjacent anchors 50a, 50b will advantageously counteract tensile forces, thus preventing the anchors 50a, 50b from separating with respect to one another. And conversely, the fixation element 10, which is fully compressed between the anchors 50a, 50b, will advantageously counteract compressive forces, thus preventing the anchors 50a, 50b from moving toward one another.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A flexible spinal fixation element, comprising:
an elongate, bioimplantable member having at least two segments that are selectively movable with respect to one another such that the elongate member is configurable in a first position, in which the segments are adapted to be angularly manipulated with respect to one another, and a second, locked position, in which the segments are aligned in a desired orientation and are immovable with respect to one another.

2. The flexible spinal fixation element of claim 1, wherein the elongate member includes a plurality of segments that are disposed around a cable member.

3. The flexible spinal fixation element of claim 2, wherein each segment includes opposed ends having surface features formed on at least a portion thereof to prevent movement between the segments when the flexible spinal fixation element is in the second, locked position.

4. The flexible spinal fixation element of claim 2, wherein each segment has a shape that is adapted to prevent movement between the segments when the segments are in the second, locked position.

5. The flexible spinal fixation element of claim 4, wherein each segment includes a female end and an opposed male end such that the female end of each segment is adapted to nest the male end of an adjacent segment.

6. The flexible spinal fixation element of claim 4, wherein each segment has a substantially tubular shape with a concave end and an opposed convex end such that the concave end of each segment is adapted to nest the convex end of an adjacent segment.

7. The flexible spinal fixation element of claim 4, wherein every other segment has a substantially spherical shape and intervening segments have a substantially tubular shape with opposed ends that are adapted to seat the spherical segments.

8. The flexible spinal fixation element of claim 7, wherein the elongate member has opposed terminal end segments, each having a substantially tubular shape.

9. The flexible spinal fixation element of claim 2, wherein the plurality of segments are adapted to be held together by a press-fit.

10. The flexible spinal fixation element of claim 2, wherein the plurality of segments are adapted to be held together by a snap-fit.

11. The flexible spinal fixation element of claim 1, wherein opposed terminal ends of the elongate member are adapted to seat a portion of a spinal anchor.

12. The flexible spinal fixation element of claim 1, wherein the elongate body includes at least two elongate segments that are mated to one another at an end thereof by a hinge.

13. The flexible spinal fixation element of claim 12, further comprising a sleeve member adapted to be disposed around the hinge to maintain the elongate body in the second, locked position.

14. The flexible spinal fixation element of claim 12, further comprising a locking mechanism adapted to mate to the hinge to maintain the elongate body in the second, locked position.

15. The flexible spinal fixation element of claim 1, wherein the elongate body comprises first and second separate segments, each segment comprising a generally elongate, hemi-spherical rod having two portions connected to one another at an end thereof by a hinge, the hinge on each of the first and second separate segments being configured to maintain the elongate body in the second, locked position when the first and second separate segments are placed together to form a cylinder.

16. A flexible spinal fixation element, comprising:
an elongate cable; and
a bioimplantable, generally elongate member slidably disposed around the cable and configurable in a first position, in which the member is adapted to be manipulated in multiple angular orientations, and a second, locked position, in which the member is fully compressed and it is immovably aligned in a desired orientation.

17. The flexible spinal fixation element of claim 16, wherein the generally elongate member comprises a bellows.

18. The flexible spinal fixation element of claim 17, wherein opposed terminal ends of the bellows are adapted to seat a portion of a spinal anchor.

19. A spinal implant kit, comprising:
a percutaneous access tube having an inner lumen extending between proximal and distal ends; and
a selectively flexible spinal fixation element configurable in a bendable position, in which the flexible spinal fixation element can be inserted through the lumen in the percutaneous access tube and angularly manipulated as it exits from the percutaneous access tube, and a locked position, in which the flexible spinal fixation element is compressed to be immovably aligned in a desired orientation.

20. The spinal implant kit of claim 19, wherein the flexible spinal fixation element comprises a plurality of segments that are adapted to form a spinal rod in the locked position.

21. The spinal implant kit of claim 20, wherein the segments are slidably disposed around a cable.

22. The spinal implant kit of claim 20, wherein each segment includes opposed ends having surface features formed on at least a portion thereof to prevent movement between the segments when the flexible spinal fixation element is in the second, locked position.

23. The spinal implant kit of claim 20, wherein each segment has a shape that is adapted to prevent movement between the segments when the segments are in the second, locked position.

24. The spinal implant kit of claim 23, wherein each segment includes a female end and an opposed male end such that the female end of each segment is adapted to nest the male end of an adjacent segment.

25. The spinal implant kit of claim 23, wherein each segment has a substantially tubular shape with a concave end and an opposed convex end such that the concave end of each segment is adapted to nest the convex end of an adjacent segment.

26. The spinal implant kit of claim 23, wherein every other segment has a substantially spherical shape and intervening segments have a substantially tubular shape with opposed ends that are adapted to seat the spherical segments.

27. The spinal implant kit of claim 19, wherein the flexible spinal fixation element includes at least two elongate segments that are mated to one another at an end thereof by a hinge.

28. The spinal implant kit of claim 19, wherein the flexible spinal fixation element comprises first and second separate, longitudinally-oriented segments, each segment having a generally hemi-spherical cross-sectional shape and including two portions connected to one another by a hinge, the hinge on each of the first and second separate segments being configured to maintain the flexible spinal fixation element in the second, locked position when the first and second separate segments are placed together to form a cylinder.

29. A method for implanting a spinal fixation element into adjacent spinal anchors disposed within vertebrae in a patient's spinal column, comprising:
introducing a flexible spinal fixation element through a percutaneous access tube coupled to a spinal anchor;
positioning the flexible spinal fixation element between the adjacent spinal anchors; and
locking the flexible spinal fixation element with respect to the adjacent spinal anchors such that the flexible spinal fixation element is compressed into an immovable configuration.

30. The method of claim 29, wherein the flexible spinal fixation element comprises a plurality of segments disposed around a cable.

31. The method of claim 30, wherein the flexible spinal fixation element is introduced through the percutaneous access tube by sliding each segment individually along the cable to form the flexible spinal fixation element as the segments are positioned between the adjacent spinal anchors.

32. The method of claim 30, wherein the step of locking the flexible spinal fixation element comprises locking the cable to the adjacent spinal anchors.

33. The method of claim 29, wherein the flexible spinal fixation element is introduced through the percutaneous access tube by sliding the fixation element along a guide wire that is positioned through the access tube.

34. The method of claim 29, wherein the flexible spinal fixation element bends as it exits the percutaneous access tube to extend between the adjacent spinal anchors.

35. The method of claim 30, wherein the step of locking the flexible spinal fixation element comprises:
positioning the cable in proximity to the adjacent spinal anchors;
compressing the segments between the adjacent spinal anchors; and
applying a closure mechanism to the each spinal anchor to lock the cable to the anchor, thereby preventing movement of the flexible spinal fixation element.

36. The method of claim 40, wherein each segment has a shape that is adapted to prevent movement between the segments when the segments are in the second, locked position.

37. The method of claim 38, wherein the flexible spinal fixation element comprises first and second elongate segments that are mated to one another at an end thereof by a hinge.

38. A method for implanting a spinal fixation element, comprising:
providing at least two spinal anchors disposed within adjacent vertebrae of a patient's spine;
providing a percutaneous access tube having an inner lumen extending between proximal and distal ends, the distal end being adapted to couple to one of the spinal anchors;
providing a flexible spinal fixation element configurable in a first position, in which portions of the flexible spinal fixation element are adapted to be angularly manipulated with respect to one another, and a second, locked position, in which the flexible spinal fixation element is compressed to be immovably aligned in a desired orientation;
inserting the flexible spinal fixation element, in the first position, through the lumen in the percutaneous access tube;
manipulating the flexible spinal fixation element to extend between the adjacent spinal anchors; and
causing the flexible spinal fixation element to be maintained in the second, locked position.

39. The method of claim 38, wherein the flexible spinal fixation element bends as it exits the percutaneous access tube to extend between the adjacent spinal anchors.

40. The method of claim 38, wherein the flexible spinal fixation element comprises a plurality of segments that are disposed around a cable member.

41. The method of claim 40, wherein the step of causing the flexible spinal fixation element to be maintained in the second, locked position comprises:
positioning the cable in proximity to the adjacent spinal anchors;
compressing the segments between the adjacent spinal anchors; and
applying a closure mechanism to the each spinal anchor to lock the cable to the anchor, thereby preventing movement of the flexible spinal fixation element.

42. The method of claim 40, wherein each segment has a shape that is adapted to prevent movement between the segments when the segments are in the second, locked position.

43. The method of claim 38, wherein the flexible spinal fixation element comprises first and second elongate segments that are mated to one another at an end thereof by a hinge.
